(19) United States
(12) Patent Application Publication
(10) Pub. No.: US 2011/0002986 A1
(43) Pub. Date: Jan. 6, 2011

(54) Stable Shellac Enteric Coating Formulation for Nutraceutical and Pharmaceutical Dosage Forms

(75) Inventors: Thomas Durig, Chadds Ford, PA (US); Yuda Zong, West Chester, PA (US)

Correspondence Address: HERCULES INCORPORATED;HERCULES PLAZA, 1313 NORTH MARKET STREET, WILMINGTON, DE, 19894-0001, US

(73) Assignee: Hercules Incorporated, Wilmington, DE (US)

(21) Appl. No.: 12/828,556

(22) Filed: Jul. 1, 2010

Related U.S. Application Data
(60) Provisional application No. 61/222,514, filed on Jul. 2, 2009.

(51) Int. Cl. A61K 9/48; A23L 1/00; A61K 47/00

(52) U.S. Cl. 424/463; 426/89; 426/531; 514/772

(57) ABSTRACT

The present invention relates to formulations for use as enteric coatings. More particularly, the present invention relates to a formulation comprising a blend of food grade ingredients that can be readily dispersed in water. This dispersion exhibits low viscosity and can easily be coated onto solid dosage forms through spraying and the like to provide an enteric coating on the solid dosage form.

### RELATED APPLICATIONS

This application claims the benefit of U.S. Provisional Application Ser. No. 61/222,514, filed on Jul. 2, 2009, the disclosure of which is incorporated herein by reference in its entirety.

### FIELD OF THE INVENTION

The present invention relates to formulations for use as enteric coatings. More particularly, the present invention relates to a formulation comprising a blend of food grade ingredients that can be readily dispersed in water and coated onto solid dosage forms to provide an enteric coating thereon.

### BACKGROUND OF THE INVENTION

Enteric film coatings are applied to oral dosage forms to delay the release of active ingredients until the dosage form has passed through the acidic environment of the stomach and has reached the near-neutral environment of the proximal small intestine. The physical chemical environment of the stomach and gastric physiology are highly variable, subject to multiple factors such as disease state, medication, age, and eating. For example in the fasted state stomach, the pH is less than 2 in healthy individuals, and gastric emptying occurs approximately every 30 minutes. However in the fed state (immediately after a meal), gastric emptying is delayed for 2 to 4 hours and gastric pH can be as high as pH 4.

It can therefore be seen that an ideal enteric coating system would have to be flexible. The majority of enterically coated dosage forms are recommended to be taken on an empty stomach. Such coatings would therefore have to be resistant to the acidic stomach environment for a relatively short time and would not be expected to be subjected to strong mechanical attrition in the stomach. On the other hand to allow for possible ingestion in the fed state, or where subsequent release from the intestine is not intended to be immediate, the coating will have to be sufficiently robust to withstand prolonged attrition in the stomach or to generally release more slowly in the alkaline environment.

There is a long history of use of enteric coatings on tablets and smaller multi-particulate dosage forms in the pharmaceutical industry. Generally polymers with acidic functional groups are chosen for enteric coatings. In the acid environment of the stomach these acid groups of the polymers are un-ionized, thus rendering the polymer water insoluble. However in the more neutral and alkaline pH of the intestine (pH 6.8-7.2), the functional groups ionize and the polymer film coating becomes water soluble.

Examples of enteric film coatings include methacrylic acid copolymers, polyvinyl acetate phthalate, cellulose acetate phtallate, hydroxypropyl methylcellulose phthalate and hydroxypropyl methylcellulose acetylsuccinate. Traditionally these water soluble coatings have been applied from organic solvent based coating solutions. However due to environmental and safety concerns and the costs associated with organic solvent coating, aqueous based dispersions and pseudo-latex systems of some of the above polymers are increasingly preferred. However, none of the above named polymers are approved for food use, including nutritional supplements, such as nutraceuticals. None of the above polymers are found in the Food Chemicals Codex (FCC) and none of the above polymers have direct food additive status or have generally regarded as safe (GRAS) status.

Several strategies have been developed to provide for food grade enteric coatings for nutraceuticals and other items classified as food.

An aqueous ethylcellulose (EC) based pseudo-latex has been used in conjunction with sodium alginate. This product is marketed as Nutrateric™ nutritional enteric coating system by Colorcon Inc. of Westpoint, Pa. This coating is supplied as a two component system in the form of an aqueous ammoniated EC dispersion with 25% solids and a separate container of sodium alginate in powder form. To prepare the final coating solution, the sodium alginate is first dispersed and dissolved in water for 60 minutes and EC dispersion is then added to the alginate solution, ensuring that the amount of water used is appropriate to achieve a final recommended dispersed solids concentration of 10% by weight. This relatively low solids concentration is recommended to ensure a sufficiently uniform coating. This relatively low solids concentration is recommended because the viscosity of this solution is inherently high. At 10% solids concentration, the coatings system has a viscosity of 430 cps at 22° C., when measured with a Brookfield Model LVT viscometer using spindle #1 at 100 rpm. For typical pumping and spraying equipment used in aqueous film coating, this is a very high viscosity and higher solids would typically be difficult to process. Such high viscosities (above 200 cps) also have a significant effect on droplet size and spreadability of the coating, thus negatively impacting film uniformity. The low solids concentration (10% by weight) is especially problematic for large scale coating of soft gelatin capsules, where prolonged exposure to high amounts of water and heat may lead to deleterious effect such as softening of the gelatin capsule walls. Furthermore, the lack of spreadability of the coating due to its relatively high viscosity can lead to blistering and non uniformity effects.

An alternative approach is the use of shellac on its own or in combination with other additives.

Shellac is a natural, food approved, resinous material obtained from the exudate of the insect Karria lacca. It is a complex mixture of materials. The two main components with enteric properties being shelloic and aleuritic acid. While shellac is well known as a material with enteric-like properties, it has a number of drawbacks. Due to insolubility in water, shellac has traditionally been used in the form of organic solvent based solutions. Additionally in its natural state, shellac is generally not soluble below a pH of 7.5 to 8.0. Rather shellac films simply soften and disintegrate after immersion in water for a number of hours. This is problematic as enteric coatings should generally be soluble or rupturable at approximately pH 6.8. Lastly shellac coatings have been reported to undergo esterification during aging, rendering the film completely water insoluble even in alkaline pH.

To obviate the use of solvents, neutralized aqueous shellac solutions are commercially available. EP 1 579 771 A1 describes a water based shellac dispersion which comprises shellac, a basic amino acid, a basic phosphate and water. The basic amino acid being selected from the group consisting of arginine, lysine and ornithine.

Several forms of aqueous ammoniated shellac dispersions are also commercially available, for example Certiseal® FC 300A film coat product, manufactured by Mantrose Haeuser, a subsidiary of RPM Corporation. Esterification of the shellac is also limited in these systems as shellac forms a salt with the ammonia or protonated amino acid.

However these systems do not address directly the need for an enteric food grade coating which is soluble or rupturable at a pH of 6.8.

In US Patent Publication 2007/0071821A, the disclosure of which is incorporated herein in its entirety, an enteric coating formulation in the form of a spray solution or suspension is disclosed. This system comprises shellac in aqueous salt form and sodium alginate, preferably in equal concentrations. An aqueous solution of an alkali salt of shellac is prepared by first dissolving the shellac in 55° C. hot water, then adding 10% ammonium hydrogen carbonate and heating to 60° C. and stirring for 30 minutes. Separately, a sodium alginate solution is prepared and the two solutions are then blended together. The system, when coated onto a dosage form rapidly disintegrates in simulated intestinal fluid (pH 6.8). However, the blend of shellac and sodium alginate as described in US Patent Publication 2007/0071821A generally has a viscosity exceeding 400 cps at a 20% solids concentration. In order to accommodate these relatively high viscosities, a relatively dilute coating solution (6-10% solids) of the shellac and sodium alginate blend have to be used to in order to facilitate spraying and pumping of the shellac and sodium alginate blend in commercially available coating equipment. Additionally, the use of an ammonium containing salt species presents various problems associated with the presence of ammonium, such as its toxicity and volatility which must be properly handled within the work site. Also, while not wishing to be bound by theory, it is believed that the volatility of the ammonium containing salt species negatively affects the shelf stability of the powder formulation using ammonium containing salt species as well as items, such as solid dosage forms, coated with enteric coatings made from the powder formulation using ammonium containing salt species.

The above approaches describe enteric coatings composed of food approved ingredients, which are either pH sensitive or more time dependant in terms of their delayed release mechanism. However, all these systems require multiple, time consuming preparation steps, often requiring two separate solutions to be made with additional dilution requirements and which increases the potential for error. Alternately, the systems require the use of pre-made dispersions of EC or shellac, which then require further dilution and blending steps thereby adding cost, complexity and/or time to the manufacturing process.

In the case of pre-made aqueous dispersions, a further cost is incurred due to the need to store and ship dispersions which contain the added bulk of water. Additionally, these pre-made aqueous dispersions require additional precautions to be taken to control microbial contamination and to minimize any physical and/or chemical instability of the dispersion.

Generally, enteric coatings are applied in relatively high amounts on a desired substrate. A five to ten (5-10%) percent weight gain during a coating step is typical. This amount of weight gain requires relatively long coating runs of two to four (2-4) hours at industry standard application rates typically used. As a point of reference, it is typical to apply aesthetic, non-functional coatings at 3% weight gain in approximately one (1) hour.

In summary, a need exists for a pH sensitive, food grade enteric coating formulation in a powder form that can be readily dispersed in water using a single, simple preparation step in as little as one (1) hour before use. A need exists for a pH sensitive, food grade enteric coating formulation which can as a dispersion be easily applied at relatively high solids (15-20%) and be readily adjusted to obtain a desired coating weight thereby allowing for more efficient coating operations. Also, a need exists for a powder form of a shellac that can be readily dispersed in water to produce coatings comprising shellac on various substrates.

### BRIEF DESCRIPTION OF THE INVENTION

The present invention relates to a formulation in powder form useful for producing a sprayable dispersion for enteric coating. The powder formulation comprising a food grade shellac, a non-ammonium alkali salt, and optionally a water-miscible polymer. The powder formulation when dispersed in water is capable of producing a sprayable dispersion for enteric coating. This coating at 15% solids in water has a viscosity of below 500 cps at about 25° C. when measured with a Brookfield LTV viscometer with a #2 spindle at 100 rpm.

A formulation for a blend of food grade ingredients that can be readily dispersed in water and the dispersion coated onto solid dosage forms to provide an enteric coating is disclosed. When dispersed in hot water, the mixture is ready for coating onto solid dosage forms, such as tablets, capsules and small particulates, after about 60 minutes of dispersing the blend into water. The resultant coating is pH sensitive. When subjected to a disintegration test in acidic simulated gastric fluid, the dosage forms coated with the inventive water dispersible powder blend resist break-up for about 60 minutes, but disintegrate within about 90 minutes after subsequent immersion in neutral (pH 6.8) simulated intestinal fluid. The water dispersible powder blend comprises shellac, non-ammonium alkali salt, and optionally a water-miscible polymer, preferably an anionic polymer such as sodium carboxymethyl cellulose (CMC), sodium alginate or pectin. Optionally, the water dispersible powder blend further comprises one or more plasticizers chosen from the group consisting of glycerine, propylene glycol, mineral oil, triacetin, polyethylene glycol, glyceryl monostearate, acetylated monoglyceride, glyceryl tricaprylate/caprate and polysorbate. Optionally, the water dispersible powder blend may comprise pigments, and detackifiers such as titanium dioxide, talc, iron oxide, and natural colors. Due to the unexpected ability to accommodate pigment loads exceeding 40% while maintaining pH sensitivity, opaque coatings on solid dosage forms with high hiding power and good “handfeel” are possible. If no pigments are included in the water dispersible powder blend of the present invention, the resultant coating is clear, translucent with a golden hue which is especially useful for coating soft gel capsules, in particular oil containing soft gel capsules such as fish oil. In this case, the enteric coating produced from the water dispersible powder blend helps prevent the premature release of fish oil in the stomach, thus reducing the chance of reflux and fish odor and after taste. When the water dispersible powder blend formulations of the present invention are dispersed in about 50 to 80° C. hot water at 15% solids concentration, they are characterized by viscosities of less than 500 cps.

The present invention also relates to an enteric coated nutraceutical or pharmaceutical solid dosage form where the enteric coated nutraceutical or pharmaceutical solid dosage form comprises a nutraceutical or pharmaceutical active ingredient and an enteric coating. The enteric coating is comprised of a food grade shellac, and a non-ammonium alkali salt.

The present invention also relates to a process for producing the sprayable dispersion for enteric coating comprising the steps of blending a food grade shellac, non-ammonium alkali salt, optionally a water-miscible polymer, one or more plasticizers chosen from glycerine, mineral oil, triacetin, polyethylene glycol, glyceryl monostearate and polysorbate, and, optionally, pigments, and detackifiers such as titanium dioxide, talc, glyceryl monostearate, iron oxides and natural colors together to form a powder formulation. The powder formulation is then dispersed in about 50 to 80° C. hot water. The dispersion is stirred for a sufficient period of time to produce a low viscosity sprayable dispersion wherein the low viscosity sprayable dispersion at 15% solids in water has a viscosity of below 500 cps at about 25° C. when measured with a Brookfield LTV viscometer with a #2 spindle at 100 rpm.

The present invention also relates to a process for producing a solid dosage form having an enteric coating and the resultant enteric coated nutraceutical or pharmaceutical wherein the above described the sprayable dispersion for enteric coating is sprayed as a low viscosity sprayable dispersion onto a nutraceutical or pharmaceutical active ingredient in a solid dosage form to produce an enteric coating on the nutraceutical or pharmaceutical active ingredient in a solid dosage form.

### DETAILED DESCRIPTION OF THE INVENTION

It has been found that food grade shellac can be blended with other food grade ingredients to form a water dispersible powder blend which is readily dispersible and useful in producing enteric coating, suitable for coating on to nutraceutical and pharmaceutical solid dosage forms, such as tablets, capsules and small particulates. In addition to shellac, the water dispersible powder blend comprises a non-ammonium alkali salt selected from the group consisting of sodium bicarbonate, sodium carbonate, potassium carbonate, potassium bicarbonate, calcium hydroxide, calcium bicarbonate and calcium carbonate, and optionally a water-miscible polymer. The water-miscible polymer is a polymer which is “food grade”, dissolvable or dispersible in water, with no discernable phase separation from the aqueous phase. Among the water-miscible polymers of use in the present invention, include alginate salt, alginic acid, proteins (e.g. wheat, soybean or corn), methylcellulose (MC), hydroxypropylcellulose (HPC), hydroxypropylmethylcellulose (HPMC), carboxymethyl cellulose (CMC), pectin, carrageenan, guar gum, locust bean gum, xanthan gum, gellan gum, arabic gum, etc. The preferred water-miscible polymers are anionic polymers such as sodium carboxymethyl cellulose (CMC), sodium alginate or pectin. Optionally, the water dispersible powder blend comprises one or more plasticizers chosen from glycerine, mineral oil, triacetin, polyethylene glycol, glyceryl monostearate, acetylated monoglyceride, glyceryl tricaprylate/caprate and polysorbate. Optionally, the water dispersible powder blend further comprises pigments, and detackifiers such as titanium dioxide, talc, iron oxide glyceryl monostearate. Additional components such as natural colors, various carbohydrate derivatives such as hypromellose, hydroxypropyl cellulose, carboxymethyl starch, carageenan and xanthan may also be used in the water dispersible powder blend of the present invention. It is preferable that the particle size of the particulate components of the water dispersible powder blend have mean diameters ranging from about 50 microns to 600 microns.

While not excluding other grades of shellac, a preferred type is Orange Dewaxed Shellac compliant with the monographs of the USP and FCC. For optimal blending and water dispersion, the shellac, in flake form, is milled prior to blending with the other ingredients of the water dispersible powder blend and resultant coating. Suitable milling and size reduction can be achieved with an impact mill for example a Fitzpatrick type hammermill. Particle size distributions where 99% of the particles by volume are smaller than 1000 microns are preferred. The amount of shellac of use in the water dispersible powder blend of the present invention is in the range of from about 20% to about 75% by weight of the blend and coating, more preferably from about 30% to about 70% by weight of the blend and coating.

The preferred water-miscible polymer for use in the water dispersible powder blend is an anionic polymer comprising sodium carboxymethyl cellulose (CMC). The preferred CMC being a low viscosity grade such as Aqualon® CMC 7L2P, marketed by Ashland Aqualon Functional Ingredients, a Business Unit of Hercules Incorporated, a subsidiary of Ashland Inc. Various grades of sodium alginate have also been found suitable for the anionic polymer for use in the water dispersible powder blend of the invention. The amount of anionic polymer of use in the water dispersible powder blend and resultant enteric coating of the present invention is in the range of from about 1% to about 18% by weight of the blend and coating, more preferably from about 2% to about 12% by weight of the blend and coating.

The water dispersible powder blend and resultant enteric coating produced therefrom also comprises an amount of a non-ammonium alkali salt. The non-ammonium alkali salt is a food grade, nonvolatile water soluble salt species which functions as a stabilizer of finished shellac coating, in addition to a basic substance to dissolve/disperse shellac. If ammonium salts alone are selected as the basic substances to dissolve/disperse shellac after accelerated aging test at 40° C. and 75% relative humidity, shellac coating may not be able to disintegrate in simulated intestine fluid (pH 6.8) within 60 minutes following 60 minute of disintegration test in simulated gastric fluid (pH 1.2).

The non-ammonium alkali salt may be any food grade, nonvolatile, water soluble inorganic or organic salt species. The non-ammonium alkali salt of use in the present invention may be selected from the group consisting of sodium, potassium, calcium, magnesium, aluminum salts. A preferred non-ammonium alkali salt comprises sodium bicarbonate. The amount of non-ammonium alkali salt of use in the water dispersible powder blend and resultant enteric coating of the present invention is in the range of from about 1.5% to about 15% by weight of the blend and coating, more preferably from about 1.5% to about 8% by weight of the blend and coating.

If the water dispersible powder blend also optionally comprises a plasticizer, the plasticizer may be selected from the group consisting of glycerine, propylene glycol, mineral oil, triacetin, polyethylene glycol, acetylated monoglyceride, glyceryl monostearate, glyceryl tricaprylate/caprate, polysorbate andoleic acid. Various edible oils may also serve as the plasticizers. The plasticizer may also be a medium-chain triglyceride which is a medium-chain (6 to 12 carbons) fatty acid ester of glycerol.

If glycerine is the plasticizer, then it may be used in an amount in the range of from about 1% to about 10% by weight of the blend, more preferably from about 2% to about 6% by weight of the blend. If mineral oil is the plasticizer, then it may be used in an amount in the range of from about 3% to about 9%, more preferably from about 5% to about 7% by weight of the blend. If glyceryl monostearate is the plasticizer, then it may be used in an amount in the range of from about 3% to about 25%, more preferably from about 5% to about 20% by weight. If polysorbate 80 is the plasticizer, then it may be used in an amount in the range of from about 0.5% to about 12%, more preferably from about 2% to about 10% by weight. If acetylated monoglyceride is the plasticizer, then it may be used in an amount in the range of from about 2% to about 12%, more preferably from about 4% to about 10% by weight.

It has also been found that glycerin monostearate also functions as an effective detackifier for the powder formulations of the present invention.

Other food grade enteric systems such as the aqueous EC pseudo latex system referred to earlier have much higher viscosities (430 cps at 10% solids by weight). Other functional enteric coating systems such as methacrylic acid co-polymer pseudo latex systems are available as low viscosity dispersions. However, none of these low viscosity enteric dispersions can be readily formed by dispersing a powder composition in water for 60 minutes prior to use using simple stirring equipment, while simultaneously meeting the requirements of a nutraceutical coating system, whose ingredients are approved as direct food additives and can be found in the FCC, the FDA direct food additive list or the FDA GRAS list The low viscosity of the dispersions of the food grade enteric system of the present invention results in excellent droplet spread ability on the dosage form substrate, resulting in smooth coatings but also high adhesion due to the ability to fill into surface imperfections and capillary pores.

Typical compositional ranges for these pigmented systems are as follows: Shellac 75-20% by weight, sodium bicarbonate 15-1.5%, CMC 18-1% by weight, if sodium alginate is included 18-1% by weight, if glycerine is included 10-2% by weight, if mineral oil is included 9-3% by weight, if glyceryl monostearate is included 25-3% by weight, if polysorbate 80 is included 12-0.5% by weight, if talc is included 60-2% by weight, if titanium dioxide is included 60-2% by weight. A more preferred range is: Shellac 70%-30% by weight, sodium bicarbonate 8-1.5% by weight, CMC 12-2% by weight, if sodium alginate is included 12-2% by weight, if glycerine is included 8-2% by weight, if mineral oil is included 7-5% by weight, if glyceryl monostearate is included 20-8% by weight, if polysorbate 80 is included 8-1% by weight, if talc is included 24-2% by weight and if TiO₂ is included 24-2% by weight.

Among the plasticizers of use in the present invention, glycerine is the most preferred due to its universal status as a food plasticizer. Furthermore, other plasticizers like triacetin, while of utility in the present invention, have surprisingly showed a potential to sometimes cause discoloration on aging. This is not seen with glycerine. For coatings that are to be applied to soft gel capsules, combinations of plasticizers are most preferred, for instance, the combination of glycerine with mineral oil or the combination of polysorbate 80 with glyceryl monostearate.

If no pigment is included in the food grade enteric system of the present invention, the resultant enteric coatings are translucent, slightly gold colored, clear coating systems which are especially useful for coating soft gel capsules.

Various effective combinations, highlighting the versatility of the system are discussed in the examples below.

The food grade enteric system in a powder form of the present invention can be manufactured by any suitable powder blending technique. Smaller lots can be readily prepared in a Cuisinart type food processor or a Hobart type planetary mixer. Larger quantities can also be manufactured in high and medium shear blenders such as, a Colette-Gral mixer, ribbon blenders and V-blenders. No blender specific issues have been identified, thus the food grade enteric system in a powder form of the present invention is expected to be able to be manufactured in a host of other blending equipment.

Typical preparation would involve any suitable powder blending technique for blending the shellac, non-ammonium alkali salt, anionic polymers, pigments, such as talc or titanium dioxide for example, for about 5 to 10 minutes, followed by addition of plasticizer over a period of about 3 to 5 minutes, after this blending may be continued for about another 3 minutes. The resulting blend is dry to the touch and can be stored in suitable containers, such as plastic lined fiber drums or boxes, until use.

When the water dispersible powder blend is dispersed in hot water, about 5° C. to 80° C., while stirring, the resulting dispersion is ready for coating pharmaceutical solid dosage forms, such as tablets, capsules and small particulates, after about sixty (60) minutes of stirring. The resultant enteric coating is pH sensitive. When soft gelatin capsules coated with the enteric coating of the present invention are subjected to a standard USP Disintegration Test in acidic simulated gastric fluid without discs, the capsules will resist break up for about sixty (60) minutes, but will rupture within about sixty (60) minutes after subsequent disintegration testing in simulated intestinal fluid (pH 6.8) without discs.

Viscosities of the dispersions were determined using a Brookfield LTV viscometer with a #2 spindle and at 100 rpm, unless noted otherwise. A low viscosity sprayable dispersion of the present invention is defined as dispersion at 15% solids in water having a viscosity of below 500 cps at 25° C. when measured with a Brookfield LTV viscometer with a #2 spindle at 100 rpm.

The examples are presented to illustrate the invention, parts and percentages being by weight, unless otherwise indicated.

### EXAMPLES

### Example 1

### Comparative

A coating formulation in the form of a sprayable aqueous dispersion was produced by weighing out the below listed amounts of polymers and ingredients and then dissolving the mixture in 65° C. water for sixty (60) minutes while strongly stirring.

The solids composition by weight without water is given below:

| | |
|---|---|
| Orange Dewaxed Shellac | 66 parts by weight |
| Ammonium carbonate | 7 parts by weight |
| CMC 7L2P | 5 parts by weight |
| Glyceryl monostearate | 8 parts by weight |
| Tween 80 | 2 parts by weight |
| Glycerin | 6 parts by weight |

When the final coating composition was applied onto fish oil capsules (˜1.8 g initial capsule weight) to a 5.8% weight gain in a O'Hara Labcoat coater with 2 kg fish oil capsule capacity, the resultant coated capsules were resistant to disintegration testing in 0.1N HCl (pH 1.2) solution for one hour, and when subsequently disintegration tested, the resultant coated capsules leaked in less than 40 minutes. After aging test at 40° C. and 75% relative humidity for 7 days, the capsules showed resistance to 0.1N HCl (pH 1.2), however some of the tested capsules did not leak within 70 minutes in the subsequent disintegration test in simulated intestinal fluid (pH 6.8).

### Example 2

To improve the disintegration of aged coated capsules, sodium bicarbonate was incorporated into the formulation to partially replace the ammonium bicarbonate. The following powder formulation was prepared as described for powder blending in Example 1:

| | |
|---|---|
| Orange Dewaxed Shellac | 68.6 parts by weight |
| Sodium bicarbonate | 4.9 parts by weight |
| Ammonium bicarbonate | 1.5 parts by weight |
| CMC 7L2P | 5.9 parts by weight |
| Glyceryl monostearate | 15.0 parts by weight |
| Tween 80 | 2.1 parts by weight |
| Acetylated monoglyceride | 2.0 parts by weight |
| (Myvacet ® 9-45 emulsifier available | |
| from Eastman Chemical Products Inc.) | |

The powder formulation was prepared as using the procedure previously described in Example 1 (Comparative). A 15% solids dispersion was made by adding the blend to 75° C. hot water while stirring for 60 minutes.

Using the same lot of fish oil soft gelatin capsules described in Example 1 (Comparative) and the same coating equipment, the soft gelatin capsules were coated to 4.0% weight gain. These coated soft gelatin capsules were found to resist to disintegration in pH 1.2 (0.1N HCl) for 1 hour, and leak within 40 minutes in simulated intestinal fluid (pH 6.8). After aging at 40° C. and 75% relative humidity for 5 days, the aged coated soft gelatin capsules showed resistance to 0.1N HC1 pH 1.2 for 1 hour, and leaked within 1 hour. Its disintegration in simulated intestinal fluid (pH 6.8) was improved, but it still delayed for 20 minutes compared to the fresh coated capsules.

### Example 3

To further increase the disintegration of aged coated capsules in simulated intestinal fluid (pH 6.8), ammonium bicarbonate was completely replaced by sodium bicarbonate. The following powder formulation was prepared using the procedure as described for powder blending in Example 1 (Comparative):

| | |
|---|---|
| Orange Dewaxed Shellac | 70 parts by weight |
| Sodium bicarbonate | 6.5 parts by weight |
| CMC 7L2P | 6 parts by weight |
| Glyceryl monostearate | 8.7 parts by weight |
| Tween 80 | 2.2 parts by weight |
| Glycerin | 6.6 parts by weight |

When coated on the same lot of fish oil gelatin capsules to a 6.5% weight gain, the coated capsules were resistant to disintegration in pH 1.2 for 1 hour and leaked in less than 20 minutes when subsequently subjected to disintegration in simulated intestinal fluid (pH 6.8). When these coated capsules were stored in 40° C. and 75% relative humidity for 14 days, they showed resistance to 0.1N HCl (pH 1.2) for 1 hour and leaked within 1 hour in the subsequent test in simulated intestinal fluid (pH 6.8). However, some coated capsules showed stickiness and severe picking was visible.

This illustrates the advantage of sodium bicarbonate in the shellac enteric coating compared to ammonium bicarbonate. The incorporation of sodium bicarbonate increased the disintegration of both fresh coated and aged capsules in simulated intestinal fluid (pH 6.8).

### Example 4

To further mitigate the stickiness of aged coated soft gelatin capsules, the following variation on Example 2 was prepared:

| | |
|---|---|
| Orange Dewaxed Shellac | 63.6 parts by weight |
| Sodium bicarbonate | 6.4 parts by weight |
| CMC 7L2P | 7.1 parts by weight |
| Glyceryl monostearate | 18 parts by weight |
| Tween 80 | 2.5 parts by weight |
| Glycerin | 2.4 parts by weight |

The powder formulation was prepared as previously described in Example 2. A 15% solids dispersion was made by adding the blend to 75° C. hot water while stirring for 60 minutes. A viscosity of 133 cps was measured for the 15% solids dispersion.

Using the same lot of fish oil soft gelatin capsules described in Example 1 (Comparative) and the same coating equipment, the soft gelatin capsules were coated to 5.5% weight gain. These coated soft gelatin capsules were found to resist to disintegration in pH 1.2 (0.1N HCl) for 1 hour, and leak within 35 minutes in simulated intestinal fluid (pH 6.8). After aging at 40° C. and 75% relative humidity for 5 days, the aged coated soft gelatin capsules showed resistance to 0.1N HCl pH 1.2 for 1 hour, and unchanged leaking time (35 minutes) in the subsequent test in simulated intestinal fluid (pH 6.8). Aging did not influence the disintegration of coated soft gelatin capsules in simulated intestinal fluid (pH 6.8) after pretreatment with 0.1N HC1 (pH 1.2) for 1 hour at 37° C.

After aging at 40° C. and 75% RH for 5 days, no severe picking was observed, compared to Example 3. This formulation had 18% (by weight) of anti-tacky agent glyceryl monostearate, instead of 8% (by weight) in Example 3.

### Example 5

The following variation on Example 4 was also prepared:

| | |
|---|---|
| Orange Dewaxed Shellac | 64.0 parts by weight |
| Sodium bicarbonate | 6.0 parts by weight |
| CMC 7L2P | 5.9 parts by weight |
| Glyceryl monostearate | 20.0 parts by weight |
| Tween 80 | 2.1 parts by weight |
| Glycerin | 2.0 parts by weight |

The powder formulation was prepared as previously described in Example 2. A 18% solids dispersion was made by adding the blend to 75° C. hot water while stirring for 60 minutes. A viscosity of 100 cps was measured for the 15% solids dispersion.

Using the same lot of fish oil soft gelatin capsules described in Example 1 (Comparative) and the same coating equipment, the soft gelatin capsules were coated to 4.3% weight gain. These coated soft gelatin capsules were found to resist to disintegration in pH 1.2 (0.1N HC1) for 1 hour, and leak within 25 minutes in simulated intestinal fluid (pH 6.8). After aging at 40° C. and 75% relative humidity for 60 days, the aged coated soft gelatin capsules showed resistance to 0.1N HCl pH 1.2 for 1 hour, and unchanged leaking time (25 minutes) in the subsequent test in simulated intestinal fluid (pH 6.8). No significant aging effect on the capsule stickiness and picking was observed for this formulation.

### Example 6

To further mitigate the stickiness of aged coated soft gelatin capsules, the following variation on Example 2 was prepared:

| | |
|---|---|
| Orange Dewaxed Shellac | 64.0 parts by weight |
| Sodium bicarbonate | 6.0 parts by weight |
| CMC 7L2P | 5.9 parts by weight |
| Glyceryl monostearate | 18.0 parts by weight |
| Tween 80 | 2.1 parts by weight |
| Glycerin | 4.0 parts by weight |

The powder formulation was prepared as previously described in Example 2. A 15% solids dispersion was made by adding the blend to 75° C. hot water while stirring for 60 minutes.

Using the same lot of fish oil soft gelatin capsules described in Example 1 (Comparative) and the same coating equipment, the soft gelatin capsules were coated to 5.2% weight gain. These coated soft gelatin capsules were found to resist disintegration in pH 1.2 (0.1N HCl) for 1 hour, and leak within 30 minutes in simulated intestinal fluid (pH 6.8). After aging at 40° C. and 75% relative humidity for 30 days, the aged coated soft gelatin capsules showed resistance to 0.1N HCl pH 1.2 for 1 hour, and unchanged leaking time (30 minutes) in the subsequent test in simulated intestinal fluid (pH 6.8). No significant difference in disintegration and no severe picking were served after aging, test at 40° C. and 75% relative humidity for 30 days.

### Example 7

The following powder formulation was prepared using the procedure as described for powder blending in Example 1 (Comparative):

| | |
|---|---|
| Orange Dewaxed Shellac | 68 parts by weight |
| Sodium bicarbonate | 6.4 parts by weight |
| Glyceryl monostearate | 19.1 parts by weight |
| Tween 80 | 2.2 parts by weight |
| Glycerin | 4.3 parts by weight |

When coated on the same lot of fish oil gelatin capsules to a 7.6% weight gain, the capsules failed to resist to leak in simulated gastric fluid (pH 1.2) for 1 hour. Further testing showed it needs about 8.9% weight gain to present resistance to simulated gastric fluid (pH 1.2) for this non-CMC formulation. In contrast, the CMC-containing formulation in Example 7 needed only about 5.2% weight gain to resist acid.

This example illustrates that the incorporation of CMC into the formulation strengthened the shellac enteric coating in acid, since the formulations in Example 6 and Example 7 had the same ratios of all other ingredients except for CMC.

### Example 8

The following formulation with pigments was made, and the coated capsules resisted simulated gastric fluid pH 1.2 for 1 hour and disintegrated in simulated intestinal fluid (pH 6.8) within 90 minutes:

| | |
|---|---|
| Orange Dewaxed Shellac | 64.0 parts by weight |
| Sodium bicarbonate | 6.0 parts by weight |
| CMC 7L2P | 5.9 parts by weight |
| Glyceryl monostearate | 18.0 parts by weight |
| Tween 80 | 2.1 parts by weight |
| Glycerin | 4.0 parts by weight |
| Titanium dioxide | 15 parts by weight |
| Talc | 15 parts by weight |

### Example 9

The following powder formulation was prepared using the procedure as described for powder blending in Example 1 (Comparative):

| | |
|---|---|
| Orange Dewaxed Shellac | 64.0 parts by weight |
| Sodium bicarbonate | 6.0 parts by weight |
| HPMC E3 | 5.9 parts by weight |
| Glyceryl monostearate | 8.0 parts by weight |
| Tween 80 | 2.1 parts by weight |
| Glycerin | 4.0 parts by weight |

When coated on fish oil gelatin capsules to a 5.7% weight gain, the capsules resisted leaking in simulated gastric fluid (pH 1.2) for 1 hour, and then leaked within 30 minutes in simulated intestinal fluid (pH 6.8). This example demonstrated that HPMC can function as a water-miscible polymer and can impart a degree of acid resistance to an enteric coating. The performance of this example was improved over the performance of Example 7 which contained no water-miscible polymer.

### Example 10

The following powder formulation was prepared using the procedure as described for powder blending in Example 1 (Comparative):

| | |
|---|---|
| Orange Dewaxed Shellac | 70.4 parts by weight |
| potasium bicarbonate | 7.6 parts by weight |
| MC A15LV | 3.0 parts by weight |
| CMC 7L2P | 3.0 parts by weight |
| Glyceryl monostearate | 12.0 parts by weight |
| Tween 80 | 2.0 parts by weight |
| Glycerin | 2.0 parts by weight |

When coated on fish oil gelatin capsules to a 5.0% weight gain, the capsules resisted leaking in simulated gastric fluid (pH 1.2) for 1 hour, and leaked in pH 6.8 buffer within 20 minutes. This experiment showed that potassium bicarbonate could also be used in enteric formulation instead of sodium carbonate or sodium bicarbonate.

### Example 11

The following powder formulation was prepared using the procedure as described for powder blending in Example 1 (Comparative):

| | |
|---|---|
| Orange Dewaxed Shellac | 55.5 parts by weight |
| Sodium bicarbonate | 5.2 parts by weight |
| Sodium alginate | 11.0 parts by weight |
| Talc | 3.9 parts by weight |
| Glyceryl monostearate | 2.0 parts by weight |
| Tween 80 | 1.8 parts by weight |
| Glycerin | 5.4 parts by weight |
| glyceryl tricaprylate (Captex ® 300 from Abitec ) | 9.2 parts by weight |
| Fumed silica | 6.0 parts by weight |

When coated on fish oil gelatin capsules to a 5.0% weight gain, the capsules resisted leaking in 0.1N HCl (pH 1.2) for 1 hour, and leaked in pH 6.8 buffer within 45 minutes.

While the invention has been described with respect to specific embodiments, it should be understood that the invention should not be limited thereto and that many variations and modifications are possible without departing from the spirit and scope of the invention.

What is claimed is:

1. A formulation in powder form useful for producing a sprayable dispersion for enteric coating, comprising:
a food grade shellac, and
a non-ammonium alkali salt.

2. The formulation in powder form of claim 1 wherein the non-ammonium alkali salt comprises a nonvolatile inorganic or organic salt.

3. The formulation in powder form of claim 1 wherein the non-ammonium alkali salt is selected from the group consisting of sodium bicarbonate, sodium carbonate, calcium hydroxide, calcium bicarbonate and calcium carbonate, potassium bicarbonate, and potassium carbonate.

4. The formulation in powder form of claim 1 wherein the non-ammonium alkali salt comprises sodium bicarbonate.

5. The formulation in powder form of claim 1 wherein the formulation in powder form further comprises a water-miscible polymer selected from the group consisting of alginate salt, alginic acid, proteins, methylcellulose (MC), hydroxypropylcellulose (HPC), hydroxypropylmethylcellulose (HPMC), carboxymethyl cellulose (CMC), pectin, carrageenan, guar gum, locust bean gum, xanthan gum, gellan gum and arabic gum.

6. The formulation in powder form of claim 5 wherein, water-miscible polymer comprises an anionic polymer selected from the group consisting of sodium carboxymethyl cellulose (CMC), sodium alginate and pectin.

7. The formulation in powder form of claim 6 wherein the anionic polymer comprises sodium carboxymethyl cellulose (CMC) in an amount in the range of from about 1% to about 18% by weight of the formulation in powder form.

8. The formulation in powder form of claim 6 wherein the anionic polymer comprises sodium alginate in an amount in the range of from about 1% to about 50% by weight of the formulation in powder form.

9. The formulation in powder form of claim 1 wherein the food grade shellac is Orange Dewaxed Shellac in an amount in the range of from about 20% to about 75% by weight of the formulation in powder form.

10. The formulation in powder form of claim 1 wherein the non-ammonium alkali salt of use in the formulation in powder form comprises in the range of from about 1.0% to about 10% by weight of the formulation in powder form.

11. The formulation in powder form of claim 1 further comprising one or more plasticizers chosen from the group consisting of glycerine, propylene glycol, mineral oil, triacetin, polyethylene glycol, glyceryl monostearate, acetylated monoglyceride, polysorbate, oleic acid, and glyceryl tricaprylate/caprate.

12. An enteric coated nutraceutical or pharmaceutical solid dosage form comprising, a nutraceutical or pharmaceutical active ingredient, and an enteric coating wherein the enteric coating comprises:
a food grade shellac, and
a non-ammonium alkali salt.

13. The enteric coated nutraceutical or pharmaceutical solid dosage form of claim 12 wherein enteric coating further comprises a water-miscible polymer selected from the group consisting of alginate salt, alginic acid, protein, methylcellulose (MC), hydroxypropylcellulose (HPC), hydroxypropylmethylcellulose (HPMC), carboxymethyl cellulose (CMC), pectin, carrageenan, guar gum, locust bean gum, xanthan gum, gellan gum and arabic gum.

14. The enteric coated nutraceutical or pharmaceutical solid dosage form of claim 13 wherein the water-miscible polymer comprises an anionic polymer selected from the group consisting of sodium carboxymethyl cellulose (CMC), sodium alginate and pectin.

15. The enteric coated nutraceutical or pharmaceutical solid dosage form of claim 14 wherein the anionic polymer comprises sodium carboxymethyl cellulose (CMC) in an amount in the range of from about 1% to about 18% by weight of the enteric coating.

16. The enteric coated nutraceutical or pharmaceutical solid dosage form of claim 14 wherein the anionic polymer comprises sodium alginate in an amount in the range of from about 1% to about 50% by weight of the enteric coating.

17. The enteric coated nutraceutical or pharmaceutical solid dosage form of claim 12 wherein the food grade shellac is Orange Dewaxed Shellac in an amount in the range of from about 20% to about 75% by weight of the enteric coating.

18. The enteric coated nutraceutical or pharmaceutical solid dosage form of claim 12 wherein the enteric coating further comprises one or more plasticizers chosen from the group consisting of glycerine, propylene glycol, mineral oil, triacetin, polyethylene glycol, glyceryl monostearate, acetylated monoglyceride, oleic acid, glyceryl tricaprylate/caprate and polysorbate.

19. The enteric coated nutraceutical or pharmaceutical solid dosage form of claim 12 wherein the enteric coating further comprises an inorganic pigment in an amount up to about 70% by weight of the enteric coating.

20. A process for producing a sprayable dispersion for enteric coating comprising the steps of:
blending a food grade shellac, a non-ammonium alkali salt, a water miscible polymer, one or more plasticizers selected from the group consisting of glycerine, propylene glycol, mineral oil, triacetin, polyethylene glycol, glyceryl monostearate, acetylated monoglyceride, glyceryl tricaprylate/caprate and polysorbate, together to form a powder formulation,
dispersing the powder formulation in about 50 to 80° C. hot water, and stirring the dispersed the powder formulation for a sufficient period of time to produce a low viscosity sprayable dispersion.

21. A process for producing a solid dosage form having an enteric coating comprising the steps of:
obtaining a nutraceutical or pharmaceutical active ingredient in a solid dosage form,
blending a food grade shellac, a non-ammonium alkali salt, a water-miscible polymer, one or more plasticizers chosen from the group consisting of glycerine, propylene glycol, mineral oil, triacetin, polyethylene glycol, glyceryl monostearate, acetylated monoglyceride, glyceryl tricaprylate/caprate and polysorbate together to form a powder formulation,
dispersing the powder formulation in about 50 to 80° C. hot water,
mixing the dispersed the powder formulation for a sufficient period of time to produce a low viscosity sprayable dispersion, and
spraying the low viscosity sprayable dispersion onto the nutraceutical or pharmaceutical active ingredient in a solid dosage form to produce an enteric coating on the nutraceutical or pharmaceutical active ingredient in a solid dosage form.
